(19) United States
(12) Patent Application Publication
(10) Pub. No.: US 2009/0068320 A1
(43) Pub. Date: Mar. 12, 2009

(54) HIGH BULK DENSITY COMPOSITIONS OF BETA-GLUCAN AND METHODS FOR MAKING THE SAME

(76) Inventor: Daniel Mark Johnson, Arlee, MT (US)

Correspondence Address: FOLEY & LARDNER LLP, 150 EAST GILMAN STREET, P.O. BOX 1497, MADISON, WI, 53701-1497, US

(21) Appl. No.: 12/205,273

(22) Filed: Sep. 5, 2008

Related U.S. Application Data
(60) Provisional application No. 60/970,568, filed on Sep. 7, 2007.

(51) Int. Cl. A23L 1/10; A23B 9/08; A61P 37/04; C08L 5/00

(52) U.S. Cl. 426/96; 426/464; 426/463; 426/622

(57) ABSTRACT

Methods for processing beta-glucan sources to obtain compositions comprising a high bulk density of beta-glucan are provided. High-bulk density compositions comprising particles of beta-glucan and products comprising the high bulk density beta-glucan compositions are also disclosed.

### CROSS-REFERENCE TO RELATED APPLICATIONS

This application claims priority to U.S. Provisional Application No. 60/970,568, filed Sep. 7, 2007, the entire contents of which are herein incorporated by reference.

### TECHNICAL FIELD

The technology relates to methods for processing beta-glucan sources to obtain a high bulk density composition of beta-glucan. The technology further pertains to high bulk density compositions comprising beta-glucan and food and nutraceutical products containing the same.

### BACKGROUND

Beta-glucan is a natural, long-chained polysaccharide found in several sources and recognized as providing benefits for coronary and glycemic health, as well as providing powerful immune-boosting properties. The term “beta” refers to the steric position of the glucose hydroxyl group involved in the chain formation. Beta-glucans can vary in chain-length (and molecular weight) and in the degree of branching. Because of their structural complexity, beta-glucan products can differ in biological activity. Beta-glucans, with (1→3)- and (1→6)-glucose links, are isolated from a variety of fungi such as shiitake (Lentinus edodes) and maitake (Grifola frondosa) mushrooms, from yeast cell walls including brewers' and bakers' yeasts (of the genus Saccharomyces), and from oat and barley bran. Mixed-linkage (1→3), (1→4) beta-D-glucan, is the predominant cell wall component of grain endosperm, particularly in oats and barley, is soluble and has a greater biological activity due to its structural branching.

Cereal grain seeds generally contain a small amount of beta-glucan, with oats and barley being recognized as the richest sources of this material. The naked oat seed, known in the art as a “groat,” typically contains from 2-4% by weight beta-glucan, depending upon the oat variety and other factors such as growing conditions. Barley seeds may typically contain twice as much beta-glucan as oat groats. Beta-glucan is generally found in higher concentrations in the outermost layers of the seed (i.e., the “bran”).

Beta-glucans are known to have a wide variety of biological activities, primarily immunological, anticarcingenic, anti-tumor, antihypercholesterolemic, digestive and glycemic activity. Beta-glucan also has immunostimulatory properties when applied topically to the skin. The antihypercholesterolemic activity of beta-glucan found naturally in oats has been acknowledged by the United States Food and Drug Administration (“FDA”). In order to receive an efficacious amount of beta-glucan for reduction of low density lipoprotein (LDL) and total serum cholesterol, total beta-glucan ingestion of at least 3 grams daily is generally recommended. For example, the FDA authorized a health claim for beta-glucan from oats and its impact on cardiovascular health and the regulatory required dose for making this claim is 750 mg of beta-glucan per serving, or 3 g per day.

### SUMMARY

In accordance with one aspect, the present disclosure provides a method for producing a particulate composition comprising beta-glucan, wherein the composition has a high-bulk density. In one embodiment, the methods may be applied to cereal bran such as e.g., barley or oat bran to obtain an oat bran concentrate. In another aspect, the method for producing a high bulk density composition of beta-glucan comprises the steps of: mixing a beta-glucan source with water in an extruder to form a hydrated beta-glucan source; extruding the hydrated beta-glucan source to form an extruded product; drying the extruded product to a moisture content of less than about 5% by weight; and milling the extruded product in at least two stages to form a high bulk density beta-glucan powder. The milling may comprise roller milling and pin milling. A fine powder having a high bulk density can be obtained by following the methods described herein.

In one aspect, the present disclosure pertains to high bulk density compositions comprising beta-glucan. In one embodiment, the high bulk density compositions are processed from various beta-glucan sources, such as cereal sources, for example oats and barley. In one embodiment, the beta-glucan source may comprise at least about 50% beta-glucan.

In one embodiment, the high bulk density compositions can be used in many applications including those in which conventional beta-glucan containing materials are used. For example, the high bulk density compositions comprising beta-glucan can be used as ingredients in foods, beverages, pharmaceuticals, nutraceuticals, topical compositions, personal care compositions and the like.

In one embodiment, the high bulk density compositions comprising beta-glucan can be incorporated into suitable dosage forms for administration to a subject. In another embodiment, the beta-glucan compositions produced by the methods described herein can be formulated for use in various industries such as in the food, beverage, nutraceutical or pharmaceutical industries.

All parts and percentages set forth in this specification are on a weight by weight basis unless otherwise specified. When referring to a numerical value, the term “about” means within plus or minus ten percent of the enumerated value, unless otherwise specified.

### BRIEF DESCRIPTION OF THE FIGURES

FIG. 1 is a line graph depicting the change in viscosity of OatVantage™ with increasing hydration at 70-80° C.

FIG. 2A is a line graph depicting the change in viscosity of OatVantage™ with increasing hydration at 91° C.

FIG. 2B is a line graph depicting the change in viscosity of Viscofiber® (oats) with increasing hydration at 91° C.

FIG. 3A depicts comparative sensory results for OatVantage™ and Viscofiber® (oats) in powder form.

FIG. 3B depicts comparative sensory results for OatVantage™ and Viscofiber® (oats) in slurry form.

### DETAILED DESCRIPTION

In one aspect, the present disclosure provides a method for producing a particulate composition comprising beta-glucan, wherein the composition has a high-bulk density. Bulk density is a property of powders, granules and other divided solids. It is defined as the mass of particles of the material divided by the total volume they occupy. The total volume includes particle volume, inter-particle void volume and internal pore volume. Bulk density is not an intrinsic property of a material; it can change depending on how the material is handled. For example, a powder poured in to a cylinder will have a particular bulk density; if the cylinder is disturbed, the powder particles will move and usually settle closer together, resulting in a higher bulk density. For this reason, the bulk density of powders is usually reported both as “freely settled” (or “loose”) and “tapped” density (where the tapped density refers to the bulk density of the powder after a specified compaction process, usually involving vibration of the container.)

The present disclosure provides methods for producing high bulk density compositions of beta-glucan comprising the steps of: mixing a beta-glucan source with water to form a hydrated beta-glucan source; extruding the hydrated beta-glucan source to form an extruded product; drying the extruded product to a moisture content of less than about 5% by weight; and milling the extruded product to form a high bulk density beta-glucan powder, wherein the milling comprises roller milling and pin milling.

In one embodiment, the beta-glucan source can be a cereal component such as e.g., cereal bran. In one aspect, the cereal bran can be selected from wheat bran, oat bran or barley bran and mixtures thereof. In another aspect, the beta-glucan source comprises oat bran concentrate, barley bran concentrate or mixtures thereof. In a specific embodiment, the beta-glucan source comprises oat bran concentrate. In one embodiment, the oat bran concentrate can be obtained by processing the source material, such as e.g., rough grind oat flakes. In a specific embodiment, the oat bran concentrate is processed from a source material comprising a low moisture content rough grind flake.

In one embodiment, the source material comprises at least about 30% by weight beta-glucan. In another embodiment, the source material comprises at least about 50% by weight beta-glucan. In yet another embodiment, the source material comprises about 50% to about 95% beta-glucan. In an illustrative embodiment, the source material comprises at least about 54% beta-glucan, or about 54% to about 95% beta-glucan.

In one embodiment, the source material comprises less than about 25% moisture content. In another embodiment, the source material comprises less than about 15% moisture content. In yet another embodiment, the source material comprises less than about 10% moisture content.

In one aspect, the first step in a method for producing a high-bulk density composition of beta-glucan comprises mixing a beta-glucan source with diluents, such as e.g., water, in an extruder to form a hydrated beta-glucan source. In one embodiment, the beta-glucan source is re-hydrated by mixing one part by weight of beta-glucan source with about 1 to about 3 parts by weight of water. In another embodiment, one part by weight of beta-glucan source is mixed with about 1 to about 2 parts by weight of water. In yet another embodiment, one part by weight of beta-glucan source is mixed with about 1.2 to about 1.5 parts by weight of water. In a specific embodiment, one part by weight of beta-glucan source is mixed with about 1.25 parts by weight of water.

In one embodiment, the step of mixing the beta-glucan source with water is conducted for about 5 min to about 120 min. This includes embodiments wherein the step of mixing the beta-glucan source with water is conducted for about 10 min to about 60 min. In an illustrative embodiment, the step of mixing the beta-glucan source with water is conducted for about 15 min to about 45 min.

In one embodiment, the step of mixing the beta-glucan source with water is conducted at ambient temperature. In an illustrative embodiment, the step of mixing the beta-glucan source with water is conducted at a temperature from about 20° C. to about 25° C. In another embodiment, the step of mixing the beta-glucan source with water is conducted at a temperature from about 15° C. to about 50° C.

In one aspect, the second step in a method for producing a high bulk density composition of beta-glucan comprises extruding the hydrated beta-glucan source to form an extruded product. In one embodiment, the hydrated beta-glucan source is extruded to form an extrudate. In one embodiment, the extrusion results in an extrudate in tubular form comprising radial projections. In one embodiment, the type of extruder employed for the extrusion process is selected from screw, screen, gear-type, low-pressure, or a pasta extruder. In a specific embodiment, the extruder employed for extrusion process is a screw-type extruder. In some embodiments, the type of extruder employed for the extrusion process is selected from a single-screw extruder, a twin-screw extruder or a continuous screw extruder. In an illustrative embodiment, the type of extruder employed for the extrusion process is a single-screw type extruder. In some embodiments the extruder is optionally fitted with a tubular die yielding a tubular form of the product with radial projections. In one embodiment, the extrusion step further comprises cutting the extruded product into sections having suitable length. In a particular embodiment, the sections have a length of about 0.1 cm to about 2.0 cm. In one embodiment, sections have a length of about 0.25 cm to about 1.5 cm.

In one aspect, the third step in a method for producing a high bulk density composition of beta-glucan comprises drying the extruded product. In one embodiment, the extruded product is dried at a temperature of no more than about 200° C. In another embodiment, the extruded product is dried at a temperature of about 120° C. to about 160° C. In yet another embodiment, the extruded product is dried at a temperature of about 135° C. to about 155° C. In an illustrative embodiment, the extruded product is dried at a temperature of about 140° C. to about 150° C. The temperature of drying is chosen so as to minimize off-notes or flavors in the finished product.

In one embodiment, the extruded product is dried for about 10 min to about 240 min. In another embodiment, the extruded product is dried for about 45 min to about 180 min. In yet another embodiment, the extruded product is dried for about 60 min to about 120 min. In an illustrative embodiment, the extruded product is dried at a temperature of about 140° C. to about 150° C. for about 60 min to about 120 min.

In one embodiment, the extruded product is dried to a moisture content of less than about 10% by weight. In another embodiment, the extruded product is dried to a moisture content of less than about 5% by weight. In yet another embodiment, the extruded product is dried to a moisture content of less than about 3% by weight.

In one aspect, the fourth step in a method for producing a high bulk density composition of beta-glucan comprises milling the extruded product. In one embodiment, the type of mill employed in the milling process is selected from a roller mill, pin mill, ball mill, hammer mill or combinations thereof. In another embodiment the milling of extruded product is a multi-step process. In one embodiment, the milling of the extruded product is conducted as a two-step process. For example, the milling step may comprise roller milling and pin milling. In one embodiment, the dried extrudate is metered into a roller mill and processed through various pairs of rollers within one or more roller mills with the same or different gap between pairs of rolls. In another embodiment, at least two of the roller pairs have different gaps. In one embodiment, the roller milling is followed by pin milling of the product. In an illustrative embodiment, the step of milling the extruded product results in a beta-glucan product which is uniform and consistent in particle size. For example, the product may have a particle size distribution wherein at least about 70% of the particles pass through a 60 mesh sieve and at least about 15% of the particles pass through a 100 mesh sieve. In another embodiment, the product may have a particle size distribution wherein at least about 70-95% of the particles pass through a 60 mesh sieve and at least about 18-40% of the particles pass through a 100 mesh sieve.

In one aspect, the fine powder oat concentrate obtained by the methods described herein is a high bulk density composition. In another aspect, the present disclosure provides high bulk density compositions comprising beta-glucan. In one embodiment, the disclosure provides compositions having a bulk density of at least about 1.0 g/ml (tapped). In another embodiment, the high density beta-glucan has a bulk density of at least about 0.75 g/ml (tapped). In yet another embodiment, the high density beta-glucan has a bulk density of at least about 0.50 g/ml (tapped). Tapped density can be measured by filling graduated cylinders with product to the 50 ml mark. The product is tapped 180 times using a STAV 2003 Stampfvolumeter. The volume measurement and weight of the product are recorded.

In a specific embodiment, the disclosure provides a high bulk density composition of beta-glucan, such as e.g., an oat bran concentrate, comprising a bulk density of at least about 0.50 g/ml (tapped). In another embodiment, the oat bran concentrate has a tapped bulk density of at least about 0.50 g/ml. In yet another embodiment, the oat bran concentrate has a tapped bulk density of from about 0.50 g/ml to about 0.90 g/ml, from about 0.75 g/ml to about 0.90 g/ml.

In one aspect, the present disclosure provides high bulk density powder in a pharmaceutically acceptable dosage form. In one embodiment, the high bulk density beta-glucan composition, including the fine powder oat concentrate processed by the method described above, may be formulated into a capsule, sachet or tablet to provide oral dosage nutraceutical products and nutritional supplements. In another embodiment, the nutraceutical products and nutritional supplements may comprise a capsule, sachet or tablet comprising the high bulk density composition, such as the oat bran concentrates described herein. In yet another embodiment, the capsule, sachet or tablet may further comprise fillers, coatings, binders, disintegrates, lubricants, processing aids, silicates, maltodextrin, starches, stearic acid, cellulose, gelatin, flavorings, colorants, sweeteners and the like. In some embodiments, stearates may be used as a processing aid.

Because of the high bulk density of the composition, greater dosages of beta-glucan can be delivered in standard capsule, tablet or sachet sizes. In one embodiment e.g., the high bulk density beta-glucan composition can be formulated into standard “0” or “00” capsules, and elongated “00” capsules, and allow FDA health claims to be asserted with a reasonable dosage, such as about 2 capsules about 4 times daily. Beta-glucan products with lower bulk density require a larger dose and/or higher number of servings per day to deliver an effective amount of beta-glucan through delivery systems such as capsules, sachets and tablets.

In one embodiment, high bulk density composition produced by the above-mentioned methods can be used in various industries such as in food, beverage, nutraceutical or pharmaceutical industry. In one embodiment, the high-bulk density composition in the form of powder or fine power may be reconstituted with liquid and ingested or used as an additive or ingredient in a product, such as a food or beverage. In some embodiments, the high bulk density compositions may be used directly as an ingredient in a product such as a food, beverage, medicine, nutraceutical, pharmaceutical and the like, and may be co-agglomerated with other products or ingredients to deliver a specific benefit. In some other embodiments, the high bulk density composition may be formulated into a topical compositions for use as an immunostimulatory agent.

As disclosed in the Examples, the high bulk density product obtained by the disclosed process has advantageous properties when compared to other similar commercially available products. The present technology, thus generally described, will be understood more readily by reference to the following examples, which are provided by way of illustration and are not intended to be limiting of the present invention.

### EXAMPLES

### Example 1

### Method for Producing High Bulk Density Beta-Glucan OatVantage™

A rough grind oat flake of 54% beta-glucan with a moisture content of less than 15% was re-hydrated by mixing with water in the ratio of one part of source material to 1.25 parts of water under mechanical agitation for 30 min. The resultant material was then extruded through a specified die yielding a tubular form with radial projections (Italpast). The product was cut in a controlled manner to a length of 0.6 cm to 1.0 cm (¼″ to ⅜″), placed on a perforated sheet and dried at 146° C. (295° F.) for a period of 90 min. The moisture content of the product was reduced to approximately 3% by weight. The dried product was then fed into a hopper and metered into a roller mill (Modem Process Equipment Corp.) at a rate of 50% (rotary paddle speed). The sections of the roller mill were set at the following distances: section 1 at 0.010″, section 2 at 0.004″, section 3 at 0.002″, and section 4 at 0.002″. The resultant powder from stage one milling was collected in a bin prior to stage two milling. The product was fed to a pin mill (Alpine), operated at 80 Hz, via a vibratory feeder set at 5 amps. A high bulk density powder, referred to herein as “OatVantage™” was obtained.

### Example 2

### Solubility of OatVantage™ and Viscofiber®

In this example, the solubility of the high bulk-density beta glucan produced as described in Example 1 was compared to Viscofiber (Natraceutical Group). The solubility at room temperature with high shear mixing was measured as follows. Briefly, 600 g solutions of both OatVantage™ and Viscofiber® (oat) at 0.6% (w/w) were prepared using water at room temperature (21° C.-25 ° C.). The solutions were homogenized at 7500 rpm for 3 minutes. The temperature of the solutions after homogenization and observations were recorded. The procedure was repeated three times for each variable. Neither produce was found to be soluble at room temperature with mixing alone.

The solubility at 91° C., 70° C.-80° C. and 40° C.-50° C. and low shear mixing measured as follows. Briefly, 600 g solutions of both OatVantage™ and Viscofiber® at 0.6% (w/w) were prepared using water at the appropriate temperature. The results are shown in Table 1. Even at boiling temperature and low concentration, Viscofiber® did not solubilize. Immediately after low shear mixing was stopped, sediment and visual particulates settled out of solution. OatVantage™ in soluble from 0.6%-1.5% at temperatures ranging from 40° C.-91° C. and low shear mixing. At higher solute concentrations, OatVantage™ was less dispersible and tended to clump, but this was overcome by mixing at low speed for 3 minutes.

TABLE 1
Solubility at 91° C., 70° C.-80° C. and 40° C.-50° C. and Low Shear Mixing

| | % Solution | | Results | | |
|---|---|---|---|---|---|
| | (w/w) | Trial # | 91° C. | 70° C.-80° C. | 40° C.-50° C. |
| OatVantage ™ | 0.6 | 1 | Soluble | Soluble | Soluble |
| | | 2 | | | |
| | | 3 | | | |
| | 1.0 | 1 | Soluble | Soluble | Soluble |
| | | 2 | | | |
| | | 3 | | | |
| | 1.5 | 1 | Soluble | Small clumps | Noticeable |
| | | 2 | | | viscous sediment |
| | | 3 | | | |
| | 2.0 | 1 | Small | Large | |
| | | 2 | Clumps | Clumps | |
| | | 3 | | | |
| | 3.0 | 1 | Large Clumps | | |
| | | 2 | | | |
| | | 3 | | | |
| Viscofiber ® | 0.6 | 3 | | Particulate | |
| | | | | settling | |

Even at boiling temperature and low concentration, Viscofiber® did not solubilize. Immediately after low shear mixing was stopped, sediment and visual particulates settled out of solution. At higher solute concentrations, OatVantage™ was less dispersible and tended to clump, but this was overcome by mixing at low speed for 3 minutes.

The solubility at 91° C., 70° C.-80° C. and 40° C.-50° C. and high shear mixing was measured as follows. Briefly, 600 g solutions of both OatVantage™ and Viscofiber® at 0.6%, 1.0%, 1.5%, 2.0% and 3% (w/w) were prepared using water at the appropriate temperature. The solution was stirred for 45 minutes while maintaining the desired temperature. The solutions were homogenized at 7500 rpm for 3 minutes. Observations were recorded. The results are shown in Table 2.

TABLE 2
Solubility at 91° C., 70° C.-80° C. and 40° C.-50° C. and High
Shear Mixing

| | | Results | | |
|---|---|---|---|---|
| | % Solution | | 70° C.- | 40° C.- |
| | (w/w) | 91° C. | 80° C. | 50° C. |
| OatVantage ™ | 0.6 | Soluble | Soluble | Soluble |
| | 1.0 | Soluble | Soluble | Soluble |
| | 1.5 | Soluble | Soluble | Viscous |
| | | | | sediment |
| | 2.0 | Soluble | Soluble | Viscous |
| | | | | sediment |
| | 3.0 | Soluble | Soluble | |
| Viscofiber ® | 0.6 | Particulates | Particulate | |
| | | suspended* | layer at the | |
| | | | top and | |
| | | | precipitation** | |
| | 1.0 | Particulates | | |
| | | suspended* | | |
| | 1.5 | Particulates | | |
| | | suspended* | | |
| | 2.0 | Particulates | | |
| | | suspended*, | | |
| | | very viscous | | |
| | 3.0 | Particulates | | |
| | | suspended*, | | |
| | | extremely | | |
| | | viscous | | |

*After a 72 hour storage time, Viscofiber ® settles out of solution and heat is necessary to resuspend the product.
**Some particulates rise and form a layer on top of the solution and some sink to the bottom immediately after processing. Further investigation at lower temperatures was not conducted as Viscofiber ® is not soluble at concentrations of 0.6% and 1.0%.

Viscofiber® was not soluble under any of the evaluated testing conditions. At room temperature, OatVantage™ was not soluble, but it was soluble at higher temperatures. At boiling temperature and low speed mixing OatVantage™ was soluble up to a 1.5% (w/w). As temperature decreases, the solubility of OatVantage™ decreases. High shear mixing (3 minutes at 7500 rpm) combined with temperatures of 75° C.-93° C. increased the solubility of OatVantage™ from 1.5% to 3.0%.

### Example 3

### Viscosity of OatVantage™ and Viscofiber® (Oat)

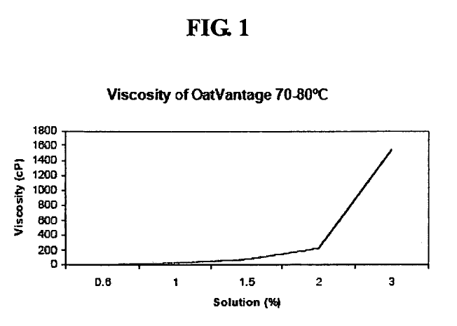

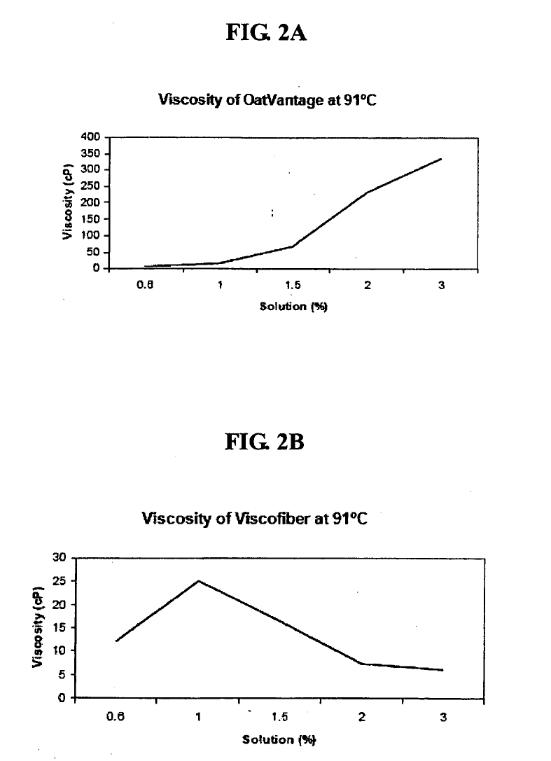

In this Example, the viscosity of OatVantage™ was compared to Viscofiber® (oat). Briefly, 0.6%, 1.0%, 1.5%, 2.0% and 3% (w/w) solutions of both OatVantage™ and Viscofiber® were prepared using water at the appropriate temperature. Solutions were stirred for 45 min and maintained at temperature range. Each solution was homogenized for 3 min at 7500 rpm and allowed to cool to room temperature. The viscosity was measured and recorded using a Brookfield DV-I+ viscometer. The results are shown in Table 3, Table 4 and FIG. 1, FIG. 2A and FIG. 2B.

TABLE 3
Viscosity at 70° C.-80° C.

| | % | | | | | |
|---|---|---|---|---|---|---|
| | Solution | | Results (cP) | | | |
| | (w/w) | Spindle | Trial #1 | Trial #2 | Trial #3 | Average |
| Viscofiber ® | 0.6 | UL | 5.11 | 5.27 | 5.33 | 5.24 |
| | 1.0 | UL | 1.93 | 1.94 | 1.90 | 1.92 |
| OatVantage ™ | 0.6 | UL | 1.85 | 1.87 | 1.93 | 1.88 |
| | 1.0 | UL | 28.80 | 28.50 | 28.30 | 28.53 |
| | 1.5 | UL | 77.60 | 79.40 | 78.70 | 78.57 |
| | 2.0 | UL | 213.20 | 215.60 | 216.50 | 215.10 |
| | 3.0 | UL | 1566.00 | 1542.00 | 1536.00 | 1548.00 |

TABLE 4
Viscosity at 91° C.

| | % | | | | | |
|---|---|---|---|---|---|---|
| | Solution | | Results (cP) | | | |
| | (w/w) | Spindle | Trial #1 | Trial #2 | Trial #3 | Average |
| Viscofiber ® | 0.6% | UL | 12.30 | 11.90 | 12.10 | 12.10 |
| | 1.0% | UL | 25.10 | 24.60 | 25.30 | 25.00 |
| | 1.5% | UL | 16.80 | 15.70 | 16.90 | 16.47 |
| | 2.0% | UL | 7.32 | 7.54 | 7.60 | 7.49 |
| | 3.0% | UL | 6.69 | 6.00 | 5.80 | 6.16 |
| OatVantage ™ | 0.6% | UL | 4.96 | 4.97 | 5.01 | 4.98 |
| | 1.0% | UL | 16.50 | 16.30 | 16.20 | 16.33 |
| | 1.5% | UL | 65.00 | 67.50 | 68.10 | 66.87 |
| | 2.0% | UL | 234.80 | 233.40 | 232.80 | 233.67 |
| | 3.0% | UL | 332.20 | 338.00 | 335.60 | 335.27 |

The results indicate that at 70-80° C. and 91° C., OatVantage™ developed higher viscosity than Viscofiber®. These findings are consistent with the solubility data described in Example 2, which indicated that OatVantage™ was more soluble compared to Viscofiber®. Upon hydration, the beta-glucan in OatVantage™ developed viscosity more readily, which can be beneficial for the delivery of health benefits.

A second assay was conducted to test the viscosity of OatVantage™ and Viscofiber®. Briefly, 1% beta-glucan solution (calculated on a dry basis) representing 1.87% OatVantage™ and 2.20% Viscofiber® were prepared. The solutions were mixed in 50 ml beakers on a heated stir plate. Heating continued at 55.7° C. with stirring for 6 hours while keeping the beakers covered to minimize water loss. Heat was turned off and stirring was maintained to allow solution to cool to about 25° C. Viscosity was measured using a Brookfield DV-I+ series viscometer. The results are shown in Table 5.

TABLE 5
Viscosity

| | Trial # | Spindle | Volume (ml) | Rpm | Viscosity (cP) |
|---|---|---|---|---|---|
| OatVantage ™ | 1 | UL | 15 | 100 | 336.4 |
| | 2 | UL | 15 | 100 | 332.6 |
| | 3 | UL | 15 | 2100 | 338.9 |
| Viscofiber ® | 1 | LV 4 | — | 100 | 2958.0 |
| | 2 | LV 4 | — | 100 | 3066.0 |
| | 3 | LV 4 | — | 100 | 3060.0 |

The results indicate that OatVantage™ was less viscous than Viscofiber® when subjected to very severe conditions such as heat for an extended period of time. These results are also consistent with the solubility findings. The beta-glucan in Viscofiber® did not hydrate readily and developed viscosity over a much longer period of time compared to OatVantage™.

### Example 4

### Comparison of Density of OatVantage™ and Viscofiber®

In this Example, the density of OatVantage™ was compared to Viscofiber®. The specification of OatVantage™ (>54% β-Glucan db) and Viscofiber® from oats (>45% β-Glucan db) are shown in Table 6.

TABLE 6
Specifications

| | | Viscofiber ®, |
|---|---|---|
| Parameter | OatVantage ™ | oats |
| Beta-glucan Concentration | ≧54% | >45% |
| (db) | | |
| Bulk Density | ≧0.65 | >0.30 |
| Specification Sheet (g/ml) | | |

Loose density was measured by filling graduated cylinders with product to the 50 ml mark without tapping. The loose weight of the powder is measured and recorded. The procedure is repeated three times. Tapped density was measured by filling graduated cylinders with product to the 50 ml mark. The product is tapped 180 times using a STAV 2003 Stampfvolumeter. The volume measurement and weight of the product were recorded. The procedure was repeated three times. The results are shown in Table 7.

TABLE 7
Results of Density Measurements

| | Loose Density (g/ml) | | | | Tapped Density (g/ml) | | | |
|---|---|---|---|---|---|---|---|---|
| Product | #1 | #2 | #3 | Average | #1 | #2 | #3 | Average |
| OatVantage ™ | 0.63 | 0.63 | 0.63 | 0.63 | 0.79 | 0.79 | 0.79 | 0.79 |
| Viscofiber ® | 0.27 | 0.28 | 0.26 | 0.27 | 0.35 | 0.35 | 0.35 | 0.35 |

The results indicate that OatVantage™ has a much higher density compared to Viscofiber®, making OatVantage™ suitable for inclusion in applications in which a small volume is desirable.

### Example 5

### Flavor Comparison of OatVantage™ and Viscofiber®

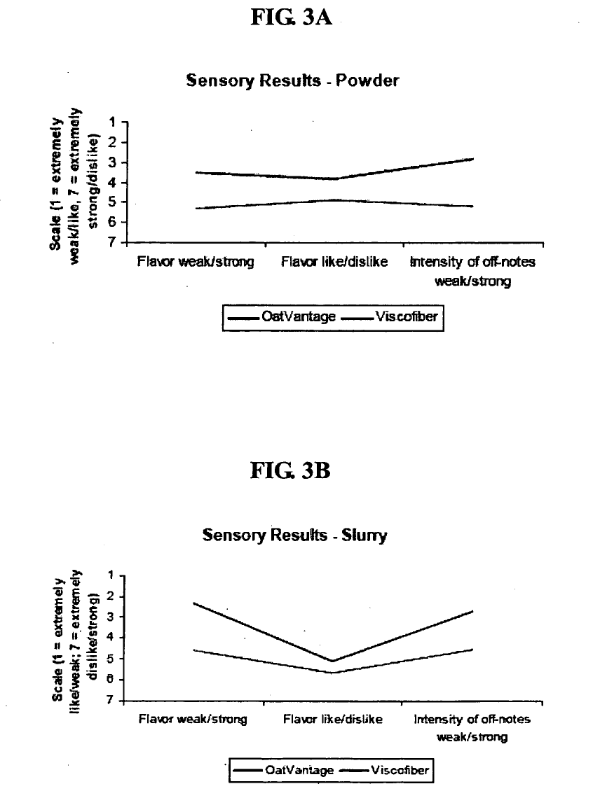

In this example, the flavor profile of OatVantage™ and Viscofiber® (oat) were compared. Ten (10) panelists were provided with a 2 g sample of each product in a powder form as well as in a 1 % beta-glucan solution (calculated on a dry basis) representing 1.87% OatVantage™ and 2.20% Viscofiber® to evaluate for the following parameters: (A) Flavor (strength and likeability); (B) Perceived Flavor Notes; and (C) Off-notes (strength). The results are shown in Table 8, Table 9, FIG. 3A, and FIG. 3B.

TABLE 8
Results of Sensory Tests -- Powder

| | Flavor | | Flavor | | Off Note Intensity | |
|---|---|---|---|---|---|---|
| | Extremely weak = 1 | | Like extremely = 1 | | Extremely weak = 1 | |
| | Extremely strong = 7 | | Dislike extremely = 7 | | Extremely strong = 7 | |
| Panelist | OatVantage ™ | Viscofiber ® | OatVantage ™ | Viscofiber ® | OatVantage ™ | Viscofiber ® |
| 1 | 4 | 6 | 5 | 6 | 3 | 7 |
| 2 | 4 | 5 | 5 | 4 | 4 | 5 |
| 3 | 5 | 5 | 5 | 4 | 5 | 5 |
| 4 | 4 | 5 | 3 | 6 | 1 | 6 |
| 5 | 4 | 5 | 4 | 4 | 4 | 5 |
| 6 | 2 | 5 | 4 | 3 | 1 | 2 |
| 7 | 2 | 5 | 4 | 6 | 4 | 5 |
| 8 | 3 | 5 | 3 | 5 | 2 | 5 |
| 9 | 3 | 7 | 2 | 7 | 1 | 7 |
| 10 | 4 | 5 | 3 | 4 | none | 5 |
| Total | 35.00 | 53.00 | 38.00 | 49.00 | 25.00 | 52.00 |
| Average | 3.50 | 5.30 | 3.80 | 4.90 | 2.78 | 5.20 |
| SD | 0.9718253 | 0.674949 | 1.0327956 | 1.286684 | 1.56347192 | 1.398412 |

| | | | |
|---|---|---|---|
| p-value | 0.0003053 | 0.0492826 | 0.00237849 |

TABLE 9
Results of Sensory Tests--Slurry

| | Flavor | | Flavor | | Off Note Intensity | |
|---|---|---|---|---|---|---|
| | Extremely weak = 1 | | Like extremely = 1 | | Extremely weak = 1 | |
| | Extremely strong = 7 | | Dislike extremely = 7 | | Extremely strong = 7 | |
| Panelist | OatVantage ™ | Viscofiber ® | OatVantage ™ | Viscofiber ® | OatVantage ™ | Viscofiber ® |
| 1 | 2 | 5 | 6 | 6 | 4 | 5 |
| 2 | 3 | 5 | 6 | 7 | 4 | 6 |
| 3 | 1 | 5 | 5 | 6 | 3 | 5 |
| 4 | 1 | 4 | 4 | 5 | 1 | 2 |
| 5 | 4 | 5 | 6 | 4 | 4 | 5 |
| 6 | 2 | 5 | 5 | 5 | 2 | 5 |
| 7 | 2 | 5 | 4 | 6 | 1 | 5 |
| 8 | 2 | 3 | 4 | 5 | 4 | 3 |
| 9 | 4 | 6 | 4 | 7 | 2 | 5 |
| 10 | 2 | 3 | 7 | 6 | 2 | None |
| Total | 23.00 | 46.00 | 51.00 | 57.00 | 27.00 | 41.00 |
| Average | 2.30 | 4.60 | 5.10 | 5.70 | 2.70 | 4.56 |
| Standard | 1.0593499 | 0.966092 | 1.1005049 | 0.948683 | 1.25166556 | 1.236033 |
| deviation | | | | | | |

| | | | |
|---|---|---|---|
| p-value | 7.792E−05 | 0.2080446 | 0.00475778 |

OatVantage™ was compared to Viscofiberg both in powder and slurry form via sensory testing. Results from both evaluations indicate that OatVantage™ had a milder flavor compared to Viscofiber®. Additionally, the perceived off-notes were rated to be significantly weaker than those present with Viscofiber®.

### Example 6

### Particle Size Distribution of OatVantage™

A characteristic particle size profile of beta-glucan particles resulted from the process of Example 1. The roller mill reduced the extruded product to a coarse powder that is very uniform and consistent in particle size. The pin mill reduces the particle size further, and also increases (broadens) the distribution of the fines. The particle size distribution allows the individual particles to fit or pack tightly together. The resulting product therefore has a high bulk density value. The particle size distribution is show in Table 10.

TABLE 10
Particle size distribution

| Mesh Size | Particle Size Distribution |
|---|---|
| +25 | 0-2% |
| −60 | 70-92% |
| −100 | 18-40% |

What is claimed is:

1. A method for producing a particulate composition comprising beta-glucan, wherein the composition has a high-bulk density, the method comprising:
(a) mixing a beta-glucan source with water to form a hydrated beta-glucan source;
(b) extruding the hydrated beta-glucan source to form an extruded product;
(c) drying the extruded product to a moisture content of less than about 5% by weight; and
(d) milling the extruded product in at least two stages to form a beta-glucan powder.

2. The method of claim 1, wherein the beta-glucan source comprises an oat bran concentrate or a barley bran concentrate.

3. The method of claim 2, wherein the beta-glucan source comprises at least about 50% by weight beta-glucan.

4. The method of claim 3, wherein beta-glucan source comprises less than about 15% moisture prior to mixing.

5. The method of claim 1, wherein the beta-glucan source is mixed with water in a ratio of about 1 part by weight of the beta-glucan source to about 1.25 parts by weight water.

6. The method of claim 1, wherein the mixing is for about 15 to about 45 minutes.

7. The method of claim 1, wherein the mixing is at a temperature from about 20° C. to about 25° C.

8. The method of claim 1, wherein the extruder is a single-screw type extruder fitted with a tubular die head.

9. The method of claim 1, wherein the extruded product is a tubular form with radial projections.

10. The method of claim 8 further comprising the step of cutting the extruded product to lengths from about 0.25 cm to about 1.5 cm.

11. The method of claim 1, wherein the step of drying is carried out at a temperature from about 135° C. to about 155° C.

12. The method of claim 1, wherein the drying is for about 1 to 2 hours.

13. The method of claim 1, wherein the drying is carried out at a temperature from about 140° C. to about 150° C. for about 1.5 hours.

14. The method of claim 1, wherein the extruded product is dried to a moisture content of less than about 3% by weight.

15. The method of claim 1, wherein the beta-glucan powder has a tapped bulk density of at least 0.50 g/mL.

16. The method of claim 1, wherein the beta-glucan powder has a tapped bulk density of at least 0.75 g/mL.

17. The method of claim 1, wherein the milling results in a product having particles, wherein at least about 70% of the particles pass through a 60 mesh sieve and at least about 15% of the particles pass through a 100 mesh sieve.

18. The method of claim 1, wherein the step of milling comprises roller milling and pin milling.

19. A high bulk density beta-glucan powder produced by the method of claim 1.

20. A capsule comprising the high bulk density beta-glucan powder of claim 19 in a pharmaceutically acceptable dosage form.
